(19) United States
(12) Patent Application Publication
(10) Pub. No.: US 2013/0095558 A1
(43) Pub. Date: Apr. 18, 2013

(54) PROCESS FOR PRODUCING RECOMBINANT HUMAN ENDOSTATIN ADENOVIRUS

(71) Applicant: Wenlin Huang, Chadds Ford, PA (US)

(72) Inventor: Wenlin Huang, Chadds Ford, PA (US)

(21) Appl. No.: 13/549,607

(22) Filed: Jul. 16, 2012

Related U.S. Application Data
(60) Provisional application No. 61/509,228, filed on Jul. 19, 2011.
(60) Provisional application No. 61/509,231, filed on Jul. 19, 2011.

(51) Int. Cl. C12N 7/02

(52) U.S. Cl. CPC C12N 7/02
USPC 435/239

(57) ABSTRACT

This invention discloses a production process for recombinant human endostatin adenovirus in order to optimize the procedure for small batch and mass industrialization. Exemplary process include steps of: (1) fermentation of eukaryotic cells (HEK293 cells) in the condition of 37° C. and 5% CO₂; (2) adenovirus infection; (3) collection of diseased cells; (4) freezing and thawing; (5) concentration by ultrafiltration; and (6) preparation and packaging of recombinant human endostatin adenovirus. The process is controllable and easy to operate. The concentration of adenovirus titers can reach 1.0×10¹²−3.0×10¹² vp/ml.

### PRIORITY CLAIMS

This application claims the benefit of priority from U.S. Provisional Application Ser. Nos. 61/509,228, filed Jul. 19, 2011, and 61/509,231, filed Jul. 19, 2011, the entire content of each of which is incorporated herein by reference in its entirety.

### TECHNICAL FIELD OF THE INVENTION

The invention generally relates to the field of bio-pharmaceutical technology. More particularly, the invention relates to novel, efficient and reliable processes for large-scale production of a recombinant human endostatin adenovirus and compositions thereof.

### BACKGROUND OF THE INVENTION

Endostatin is a naturally occurring antiangiogenic protein that is believed to inhibit the formation of blood vessels that feed tumors. It was first discovered in the Children's Hospital Boston laboratory of Judah Folkman. As an endogenous angiogenesis inhibitor, endostatin may interfere with the pro-angiogenic action of growth factors such as basic fibroblast growth factor (bFGF/FGF-2) and vascular endothelial growth factor (VEGF). (Folkman, J. (2006) Exp. Cell. Res. 312 (5): 594-607.)

Endostatin was first found secreted in the media of non-metastasizing mouse cells from a hemangioendothelioma cell line and was subsequently found in humans. Endostatin was reported to play a role of the ECM in suppression of neoangiogenesis. (O'Reilly, et al. (1997) Cell 88: 277-85; Standker, et al. (1997) FEBS Lett. 420: 129-33.)

Endostatin has been identified as a C-terminal fragment of Collagen type 18. Endostatin has a short half-life and its therapeutic effect is dose-dependent. Without wishing to be bound by the theories, endostatin represses cell cycle control and anti-apoptosis genes in proliferating endothelial cells, resulting in cell death. (Shichiri, et al. (2001). FASEB J 15: 1044-53.) Endostatin blocks pro-angiogenic gene expression controlled by c-Jun N terminal kinase (JNK) by interfering with TNFα activation of JNK. (Yin, et al. (2002) Mol. Ther. 5: 547-54.) It reduces the growth of new cells by inhibiting cyclin D1. As a result, cells arrest during G1 phase and enter apoptosis. (Dhanabal, et al. (1999) Biochem Biophys Res. Comm. 258: 345-52; Hanai, et al. (2002) J. Biol. Chem. 277: 16464-9.)

Endostatin has several advantages in its use in cancer therapy. First of all, endogenous endostatin has shown little or no resistance or toxicity in humans compared to other cancer drugs. Endostatin has been estimated to affect about 12% of the human genome and a broad spectrum of potential activity as compared to single-molecule therapies.

Adenoviruses are viruses that carry their genetic material in the form of double-stranded DNA. When adenoviruses infect a host cell, they introduce their DNA molecule into the host. The genetic material of the adenoviruses is not incorporated into the host cell's genetic material. The DNA molecule is left free in the nucleus of the host cell, and the instructions in this extra DNA molecule are transcribed just like any other gene. The only difference is that these extra genes are not replicated when the cell undergoes cell division so the descendants of that cell do not have the extra gene. As a result, treatment with the adenovirus will require re-administration in a growing cell population.

Human endostatin adenovirus is a replication-defective, recombinant oncolytic adenovirus encoding human endostatin. Upon intratumoral administration, the adenovirus infects and replicates in tumor cells. The expressed endostatin may inhibit endothelial cell proliferation and angiogenesis which may result in a reduction or elimination of tumor growth.

Cancer therapy with recombinant endostatin protein, however, has been hampered by its shot half-life, difficulties in protein production and stability issues of long-term storage of bioactive protein. Recombinant human endostatin adenovirus has proven expensive and difficult to produce in large scale. There is a pressing need for efficient and inexpensive production processes that provide consistently high quality recombinant human endostatin adenovirus.

### SUMMARY OF THE INVENTION

The invention provides a novel process suitable for large-scale production of recombinant human endostatis adenovirus for human therapeutic use, particularly, for cancer treatment. The production processes of the invention are efficient, easily controlled, stable as well as inexpensive compared to conventional methods.

In one aspect, the invention generally relates to a process for producing recombinant human endostatin adenovirus useful for injective administration to human. The process includes: fermenting eukaryotic cells in a culture; performing adenovirus infection of the eukaryotic cells to yield diseased eukaryotic cells having recombinant human endostatin adenovirus; harvesting the diseased eukaryotic cells; causing cell lyses of the diseased eukaryotic cells; and purifying the resulting recombinant human endostatin adenovirus. In certain preferred embodiments, the eukaryotic cells are Human Embryonic Kidney 293 (HEK293) cells.

In certain preferred embodiments, cell lyese is caused by freezing-thawing of the diseased eukaryotic cells.

In certain preferred embodiments, purifying the resulting recombinant human endostatin adenovirus includes the steps of: isolating the human endostatin adenovirus by chromatography; and separating the human endostatin adenovirus by centrifugation or ultra-filtration.

In another aspect, the invention generally relates to a process for large-scale production of recombinant human endostatin adenovirus. The process includes:
fermenting HEK293cells in a culture under conditions of: a cell density from about 2×10⁵ mL to about 5×10⁵/mL, a temperature from about 36° C. to about 37° C., a CO₂ concentration of about 5%, a pH in the range from about 7.2 to about 7.4, a solvent oxygen concentration of about 30% to about 70%, and a stirring speed of about 30 rpm to about 100 rpm;
performing adenovirus infection of HEK293 cells to yield diseased eukaryotic cells having recombinant human endostatin adenovirus, wherein the adenovirus infection is carried out to a MOI in the range from about 10 to about 30 and under conditions of a pH in the range from about 7.0 to about 7.4, a temperature from about 36° C. to about 37° C., and a dissolved oxygen concentration of about 30% to about 70%;
harvesting the diseased HEK293 cells in about 48 to about 72 hours from virus infection;
causing cell lyses by at least three cycles of freezing-thawing of the diseased HEK293 cells;
isolating the human endostatin adenovirus by anion exchange chromatography carried out at a pH from about 7.5 to about 8.5 with a buffer comprising from about 1 to about 100 mmol/L of phosphate, a Tris solution, from about 0.1 mmol/L to about 50 mmol/L of MgCl₂, from about 1 mmol/L to about 2000 mmol/L of NaCl, and from about 1% to about 15% of glycerol;
separating the human endostatin adenovirus by ultra-filtration with a 0.22 μM filter; and
allocating the purified recombinant human endostatin adenovirus into aliquots comprising human endostatin adenovirus from about 1.0×10¹² VP/mL to about 3.0×10¹² VP/mL.

### BRIEF DESCRIPTION OF THE DRAWINGS

FIG. 1 shows a schematic illustration of an embodiment of the invention for production of recombinant human endostatin adenovirus injection.

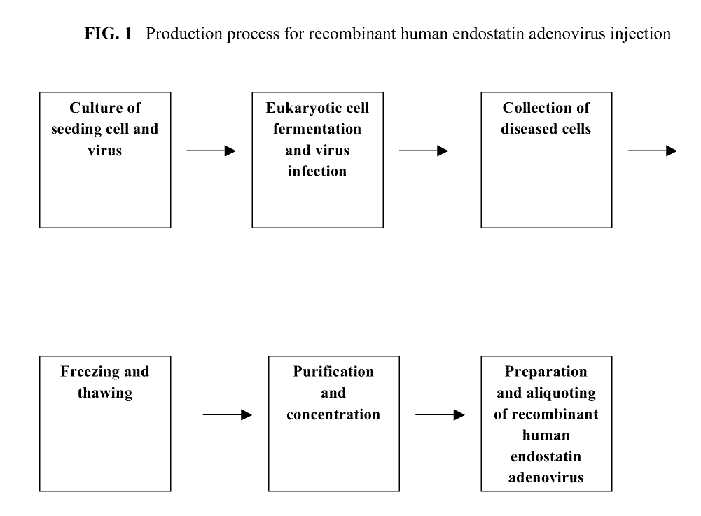

FIG. 2 shows an exemplary photograph of certain normal HEK293 cells used in this invention.

FIG. 3 shows an exemplary electron micrograph of adenovirus used in this invention.

FIG. 4 shows an exemplary photograph of certain diseased HEK293 cells.

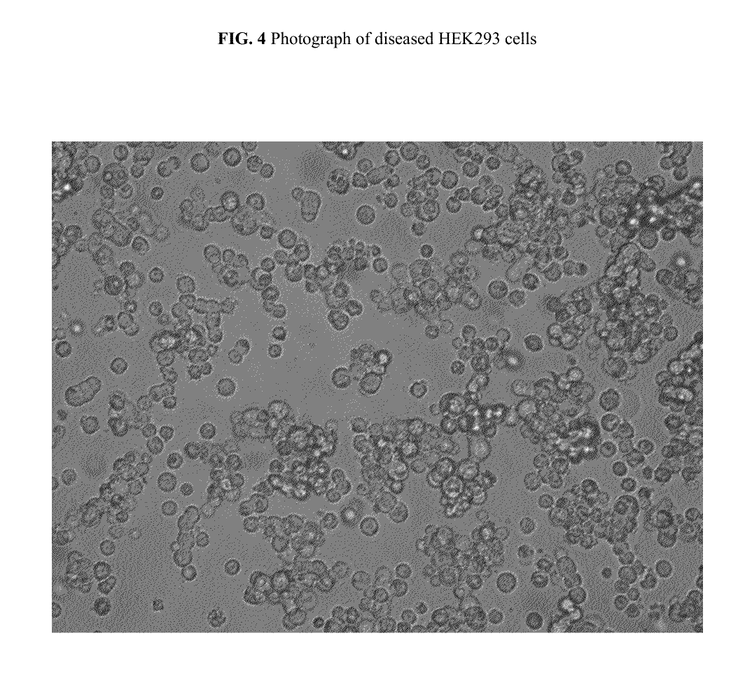

FIG. 5 shows exemplary chromatography data of recombinant human endostatin adenovirus after one-step purification.

FIG. 6 shows exemplary HPLC data of recombinant human endostatin adenovirus.

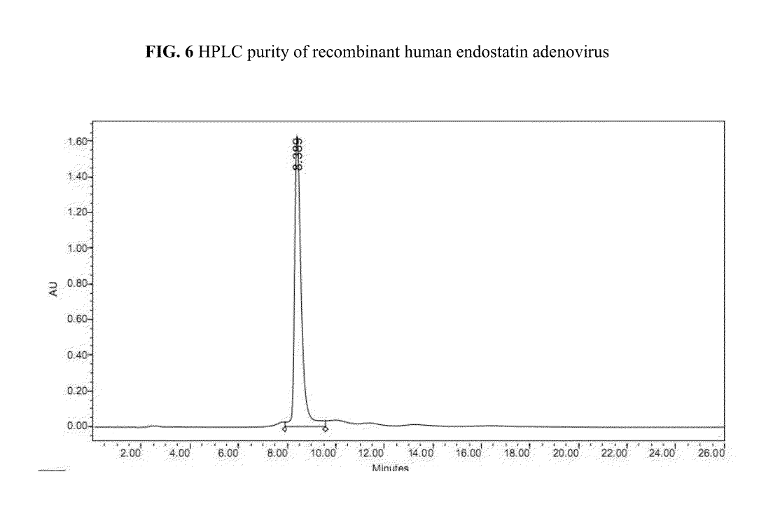

FIG. 7 shows exemplary comparison of tumor weight between the groups of mice receiving Ad-rhE, Ad-lacZ or DMEM

FIG. 8 shows exemplary concentration data of endostatin in mice receiving Ad-rhE, Ad-lacZ or DMEM.

### DETAILED DESCRIPTION OF THE INVENTION

The invention is based, in part, on a novel process suitable for large-scale production of recombinant human endostatis adenovirus for human therapeutic use. The process of the invention is efficient, easily controlled, stable and inexpensive compared to current methods.

Compared to non-viral or lentiviral vectors, adenoviruses vector offer wide a host range, low pathogenicities to human, the ability to effectively proliferate and high titer. In addition, adenovirus is not integrated into chromosome, does not cause insertional mutations and can be proliferated in suspension culture medium. In recent years, adenoviral vectors have attracted increasing attention and have become one of the main delivery systems for tumor gene therapy.

In certain preferred embodiments, the processes of the invention employ a second-generation adenovirus vector in which the E2A gene was modified to introduce a temperature-sensitive mutation in order to reduce vector immunogenicity. This improved adenovirus vector can produce high titer recombinant adenovirus and increase expression of biologically active human endostatin in a variety of cells.

Expressed human endostatin can inhibit endothelial cell proliferation and tumor angiogenesis by blocking tumor blood supply, which specifically inhibits tumor growth and induces apoptosis of tumor cells.

Natural resource of endostatin is limited. Recombinant products of endostatin are normally expressed and extracted from E. coli. However, highly expressed proteins form inclusion bodies in prokaryotic cells, causing difficulties for purification. In addition, post-translational processing and modification of prokaryotic expression system is not well developed, leading to a low biological activity of expressed products.

Production and purification of recombinant human endostatin adenovirus is the key to promote gene therapy. Currently, the cells are grown in tissue culture flask and “cell factory” and the infected cells are collected. The stock solution of virus is obtained after freezing-thawing and two times of CsCl gradient ultracentrifugation. Studies have shown that the virus yield from 100 single-tray cell factory is approximately 6×10¹² PFU. Purification through CsCl gradient centrifugation has many limitations and thus cannot meet the requirement of the adenovirus for gene therapy. In addition, CsCl gradient centrifugation is laborious and time-consuming. Therefore, alternative methods for purification other than CsCl gradient ultracentrifugation need to be developed. Very few reports are available on the purification of virus by chromatography. Although chromatography has been widely used for purification of recombinant proteins, it is particularly important to apply chromatography in purification of adenovirus vectors.

This invention develops and optimizes the processes for eukaryotic cell fermentation, adenovirus infection, proliferation, harvesting and purification. The invention provides an industrial-scale process that satisfies the GMP requirement for production of recombinant adenovirus. Ultimately, it enables a production system for commercialization of recombinant adenovirus as genetic drugs.

The eukaryotic cell fermentation system (HEK293 cell system) described in this invention overcomes these disadvantages. The system contains transcriptional and translational system and strictly controls gene expression resulting in high expression of target gene. Folding, phophosphorylation and glycosylation of human endostatin expressed from the eukaryotic system of the invention are of much nigher quality than that expressed from prokaryotic systems. The expressed human endostatin has relatively high biological activity and can directly inhibit tumor growth.

In an exemplary embodiment of the invention, HEK293 cell is used as the host to produce recombinant adenovirus. NBS bioreactor is used as suspension or fixed culture system. The flowing speed of the medium can be adjusted according to the designed parameters. Diseased cells containing large amount of recombinant human endostatin adenovirus are harvested. After freezing and thawing, recombinant human endostatin adenovirus is purified by one-step chromatography and the stock injection solution is obtained. The stock solution is filtrated to remove the bacteria and the final products are obtained after aliquoting.

The process disclosed herein can effectively increase the ratio activity and purification efficacy of adenovirus and reduce the production cycle and product costs. In vitro and in vivo studies showed that the quality of the products produced by processes of the invention can be effectively controlled. The target gene can be highly expressed in vivo and in vitro. Expression of target gene significantly inhibited the proliferation of vascular endothelial cells and effectively inhibited the growth of transplanted tumors in nude mice. Injected recombinant adenovirus was mainly distributed in the local site of tumors. No significant acute toxicity was observed.

In one aspect, the invention generally relates to a process for producing recombinant human endostatin adenovirus useful for injective administration to human. The process includes: fermenting eukaryotic cells in a culture; performing adenovirus infection of the eukaryotic cells to yield diseased eukaryotic cells having recombinant human endostatin adenovirus; harvesting the diseased eukaryotic cells; causing cell lyses of the diseased eukaryotic cells; and purifying the resulting recombinant human endostatin adenovirus.

In certain preferred embodiments, the eukaryotic cells are Human Embryonic Kidney 293 (HEK293) cells.

In certain preferred embodiments, cell lyese is caused by freezing-thawing of the diseased eukaryotic cells. Other methods for cell lysis include hypotonic solution, hypertonic solution, freezing and thawing, sonication, current and microfluidc technology or non-ionic detergents (e.g., Tween-20, Triton X-100).

In certain preferred embodiments, fermenting eukaryotic cells is carried out in a DMEM medium comprising glucose at a concentration greater than about 1 g/L (e.g., greater than about 1.0 g/L, 1.1 g/L, 1.2 g/L, 1.3 g/L), fetal bovine serum concentration from about 8% to about 12% (e.g., about 8.0%, 9.0%, 10.0%, 11%, 12.0%), and an adenovirus culture medium with about 4% to about 6% serum (e.g., about 4.0%, 4.5%, 5.0%, 5.5%, 6.0%). The fermentation may be carried out in a NBS bioreactor for cell culture.

In certain preferred embodiments, fermentation of eukaryotic cells is performed under such conditions: at a cell density from about 2×10⁵ mL to about 5×10⁵/mL (e.g., about 2.0×10⁵ mL, 2.5×10⁵ mL, 3.0×10⁵ mL, 3.5×10⁵ mL, 4.0×10⁵ mL, 4.5×10⁵ mL, 5.0×10⁵ mL), at a temperature from about 36° C. to about 37° C. (e.g., about 36.0° C., 36.5° C., 37.0° C., 37.5° C.), a CO₂ concentration of about 4% to about 6% (e.g., about 4.0%, 4.5%, 5.0%, 5.5%, 6.0%), with a pH in the range from about 7.2 to about 7.4 (e.g., about 7.2, 7.3, 7.4), with a dissolved oxygen concentration of about 30% to about 70% (e.g., about 30%, 40%, 50%, 60%, 70%), and at a stirring speed of about 30 rpm to about 100 rpm (e.g., about 30 rpm, 40 rpm, 50 rpm, 60 rpm, 70 rpm, 80 rpm, 90 rpm, 100 rpm).

In certain preferred embodiments, fermentation of eukaryotic cells is performed with a gradual increase in a medium perfusion rate according to glucose consumption during the culturing process to maintain a concentration of glucose to greater than about 1 g/L (e.g., greater than about 1.0 g/L, 1.1 g/L, 1.2 g/L, 1.3 g/L).

In certain preferred embodiments, adenovirus infection of the eukaryotic cells is carried to a MOI in the range from about 10 to about 30 (e.g., about 10, 15, 20, 25, 30) and harvesting the diseased eukaryotic cells is carried out after from about 48 to about 72 hours (e.g., about 48, 54, 60, 66, 72 hours) of virus infection. And adenovirus infection of the eukaryotic cells is carried to with a pH in the range from about 7.0 to about 7.4 (e.g., 7.0, 7.1, 7.2, 7.3, 7.4), at a temperature from about 36° C. to about 37° C. (e.g., about 36.0° C., 36.5° C., 37.0° C., 37.5° C.), and with an oxygen concentration of about 30% to about 70% (e.g., about 30%, 40%, 50%, 60%, 70%).

In certain preferred embodiments, the diseased eukaryotic cells are harvested when about 92% to 97% (e.g., about 92%, 93%, 94%, 95%, 96%, 97%) of the eukaryotic cells have been infected by adenovirus.

In certain preferred embodiments, purifying the resulting recombinant human endostatin adenovirus includes: isolating the human endostatin adenovirus by chromatography; and separating the human endostatin adenovirus by centrifugation or ultra-filtration.

In certain preferred embodiments, the process further includes allocating the purified recombinant human endostatin adenovirus into aliquots. In certain preferred embodiments, the aliquots comprise human endostatin adenovirus at about from about 1.0×10¹² to about 3.0×10¹² vp/ml (e.g., (1±0.1)×10¹² VP/mL, (1.5±0.1)×10¹² VP/mL, (2±0.1)×10¹² VP/mL, (2.5±0.1)×10¹² VP/mL, and (3±0.1)×10¹² VP/mL).

In certain preferred embodiments, isolating the human endostatin adenovirus by chromatography comprises using anion exchange fillings selected from Q Sepharose XL Virus Licensed, Q Sepharose XL, DEAE-Sephacel and DEAE-Biogel P for chromatography.

In certain preferred embodiments, chromatography is carried out at a pH from about 7.5 to about 8.5 (e.g., about 7.5, 7.7, 7.9, 8.1, 8.3, 8.5), in a buffer comprising from about 1 mmol/L to about 100 mmol/L (e.g., 1.0 mmol/L, 5.0 mmol/L, 10 mmol/L, 20 mmol/L, 50 mmol/L, 100 mmol/L) of phosphate, a Tris solution, from about 0.1 mmol/L to about 50 mmol/L (e.g., about 0.1 mmol/L, 0.5 mmol/L, 1.0 mmol/L, 5.0 mmol/L, 10 mmol/L, 20 mmol/L, 50 mmol/L) of MgCl₂, from about 1 mmol/L to about 2000 mmol/L (e.g., 1 mmol/L, 10 mmol/L, 100 mmol/L, 500 mmol/L, 1000 mmol/L, 2000 mmol/L) of NaCl, and from about 1% to about 15% (e.g., about 1%, 3%, 5%, 8%, 10%, 12%, 15%) of glycerol.

In certain preferred embodiments, chromatography is carried out at a sample loading speed from about 10 to 20 mL/min (e.g., about 10 mL/min, 12 mL/min, 14 mL/min, 16 mL/min, 18 mL/min, 20 mL/min) and an elution speed from about 15 to about 25 mL/min (e.g., 15 mL/min, 18 mL/min, 20 mL/min, 22 mL/min, 25 mL/min). In certain preferred embodiments, ultrafiltration is carried out with a 0.22 μM filter although other similar size filters may be used.

In another aspect, the invention generally relates to a process for large-scale production of recombinant human endostatin adenovirus. The process includes:
fermenting HEK293cells in a culture under conditions of a cell density from about 2×10⁵ mL to about 5×10⁵/mL, a temperature from about 36° C. to about 37° C., a CO₂ concentration of about 5%, a pH in the range from about 7.2 to about 7.4, a solvent oxygen of about 30% to about 70%, and a stirring speed of about 30 rpm to about 100 rpm;
performing adenovirus infection of HEK293 cells to yield diseased eukaryotic cells having recombinant human endostatin adenovirus, wherein the adenovirus infection is carried out to a MOI in the range from about 10 to about 30 and under conditions of a pH in the range from about 7.0 to about 7.4, a temperature from about 36° C. to about 37° C., and a solvent oxygen of about 30% to about 70%;
harvesting the diseased HEK293 cells from about 48 to about 72 hours of virus infection;
causing cell lyses by at least three cycles of freezing-thawing of the diseased HEK293 cells;
isolating the human endostatin adenovirus by anion exchange chromatography carried out at a pH from about 7.5 to about 8.5 with a buffer comprising from about 1 to about 100 mmol/L of phosphate, a Tris solution, from about 0.1 to about 50 mmol/L of MgCl₂, from about 1 to about 2000 mmol/L of NaCl, and from about 1% to about 15% of glycerol;
separating the human endostatin adenovirus by ultra-filtration with a 0.22 μM filter; and
allocating the purified recombinant human endostatin adenovirus into aliquots comprising human endostatin adenovirus at from about 1.0×10¹² to about 3.0×10¹²vp/ml.

In certain preferred embodiments, anion exchange chromatography uses fillings selected from Q Sepharose XL Virus Licensed, Q Sepharose XL, DEAE-Sephacel and DEAE-Biogel P for chromatography.

Activity of recombinant human endostatin adenovirus is shown to be excellent with a specific activity of 4.5% -7%, for example.

### EXAMPLES

The representative examples are intended to help illustrate the invention, and are not intended to, nor should they be construed to, limit the scope of the invention.

### 1. Cell Fermentation and Virus Infection

One vial of HEK293 frozen cells (14^{th} generation) was recovered using DMEM supplemented with 10% fetal bovine serum (FBS) and grown in 37° C. incubator with 5% CO₂. The cells were passed once every 3-4 days. Once 20 flasks of cells were obtained, cells were transported into NBS 5L-cell fermentation tank. The cell density in the fermentation tank was adjusted to 2×10⁵−5×10⁵/mL, and the temperature was maintained at 36° C.-37° C. Dissolved oxygen was maintained at 30%-70% using oxygen and nitrogen.

After 1-5 days of inoculation, CO₂ was used to control pH at 7.2-7.4 and in the later period, NaOH was used to control pH at 7.2-7.4. In the early period (1-5 days after inoculation), the stirring speed was controlled at 40-80 rpm, while in the later period, the stirring speed was controlled at 60-120 rpm. Supernatant was collected every day from the fermentation tank to determine the content of glucose in order to calculate the consumption of glucose by the cells each day. When the glucose content was reduced to 1 g/L, perfusion culturing was performed. The perfusion speed was increased to 0.15-2.5 volume/day based on the increased consumption of glucose each day. The glucose concentration in the fermentation tank was maintained above 1 g/L. The cells were cultured continuously in the fermentation tank for 10 to 15 days.

When the glucose consumption reached 35-55 g/day, the cell amount required for virus infection was achieved. DMEM supplemented with 5% FBS was used to replace 50%-80% of the medium in the fermentation tank. The cells were then infected with recombinant endostatin adenovirus at MOI (Multiplicity Of Infection=ratio of infectious virus particles to cells) of 10-30. Temperature was controlled at 37° C., pH was controlled at 7.0-7.4, the stirring speed was set at 60-120 rpm and the dissolved oxygen was controlled at 30-70% as described above. Supernatant was collected every 12 hours from the fermentation tank to determine the content of glucose. Based on the glucose consumption, the perfusion speed was adjusted to 1.5-3 volume/day. The cells were grown for 48-72 hours and the human endostatin was determined in the supernatant. The curve of endostatin expression was obtained. When the expression level was initially increased and then decreased, the diseased cells were harvested.

2. Collection of diseased cells

HEK293 cells were monitored after virus infection. When 95% of the cells were diseased, cells were harvested.

3. Freezing and thawing of the cells

Cell lysis was by freezing and thawing of the cells. Cells were precipitated and placed in −80° C. Once the cells were completely frozen, they were transported to a 37° C. water bath. This procedure was repeated for 3 times. Lysis buffer A was added to the host cells containing adenovirus at a ratio of 1:1-1:1.5 (cell: buffer). Lysis buffer A contained 1-100 mM Tris, 0.5-50 mM MgCl₂, 1-500 mM NaCl with a pH of 7.5-8.5. The cell debris can be removed by centrifugation or ultrafiltration. Adenovirus was remained in the supernatants or ultrafiltrates.

4. Purification (one-step chromatography)

Q Sepharose XL was used as anion exchange, low pressure chromatography fillings. Linear flow rate was 100 cm-700 cm/h. For column regeneration, 0.05-1 mol/L NaOH solution with a 0.5-3 volumes of the column (linear flow rate: 20 cm-100 cm/h) was used. Pyrogen and other impurities were removed and the column was washed with water to neutral condition. Buffer A (1-5 volume of the column) was used to balance the column. The recipe for buffer A was 1-100mM Tris, 0.5-50 mM MgCl₂, 3-10% glycerol and pH 7.5-8.5. For sample loading, 0.45-0.65 μM membrane was used to remove the cell debris from the adenovirus solution. Buffer A (3-5 volume of the column) was used to balance the column. Gradient or isocratic buffer B (3-5 volume of the column) containing 0.1-0.4 mol/L salt was used to wash impurities (mainly to remove the protein and nucleic acid peak). Buffer B contained 1-100 mM Tris, 0.5-50 mM MgCl₂, 1-2000 mM NaCl, 3-10% Glycerol with a pH of 7.5-8.5. Gradient or isocratic buffer B containing 0.1-1.0 mol/L salt was used to elute adenovirus. Elution containing virus peak was collected.

5. Preparation of recombinant human endostatin adenovirus injection solution

Ultrafiltration was used to concentrate the virus. Virus preservation solution was used to replace the high salt buffer. Desalted ultrafiltrates were collected as crude virus solution. After removal of bacteria by 0.22 μM filter, preservative solution was added and virus was diluted to (1±0.1)×10¹² VP/ml. All the procedures were conducted under low temperature condition (e.g., 4° C.) except those that require special temperature.

### Example 1: Production of recombinant human endostatin adenovirus

Experimental materials: Human kidney epithelial cells (HEK293 cells) were purchased from ATCC.

### Main Instruments and Equipments:

A: Water equipment: Pure water producing equipment was provided by Water Treatment Equipment Co., Ltd. of China. Injection water producing equipment was provided by Jilin Huatong Pharmaceutical Equipment Co., Ltd. The capacity of water production was 500 kg/h. pH of the water was 6.0-7.0. The produced water contains less than 0.5 EU/mL endotoxin. These parameters meet water requirements.

B: Cell culture: CO₂ incubator was provided by Hearus (Germany).

C: Bioreactor: Basket stirring bioreactor was provided by NBS (US).

D: Purification equipment: Purifier 100 was provided by GE.

E: Packaging equipment: All types of pipettes were provided by Eppendorf.

F: Desktop capping machine (Type: 6TI12) was provided by Nanjing Animal husbandry and medical equipment factory.

### Main Reagents:

| Reagents | Vendor | Notes |
|---|---|---|
| DMEM | GIBCO BRL | Powder |
| Fetal bovine serum | GIBCO BRL | |
| Trypsin | GIBCO BRL | Cell culture grade |
| Glycerol | Hunan ErKang | Pharmaceutical grade |
| | Pharmaceutical Co., Ltd. | |
| Injection water | | Made in-house |
| Tris | Suzhou Yake Chemical | Pharmaceutical grade |
| | Reagent Co., Ltd. | |
| NaCI | Hebei Huacheng | Pharmaceutical grade |
| | Pharmaceutical Co., Ltd. | |
| MgCI₂ | Beijing Yanjing | Pharmaceutical grade |
| | Pharmaceutical Co., Ltd. | |
| HCI | Chengdu Hua Yi | Pharmaceutical grade |
| | Pharmaceutical Excipients | |
| | Manufacturing Co., Ltd. | |

Treatment of equipments: All the equipments were sterilized and endotoxin was removed.

Ingredients of medium: DMEM contains high glucose, NaHCO₃ and 10% FBS.

### Experimental Methods:

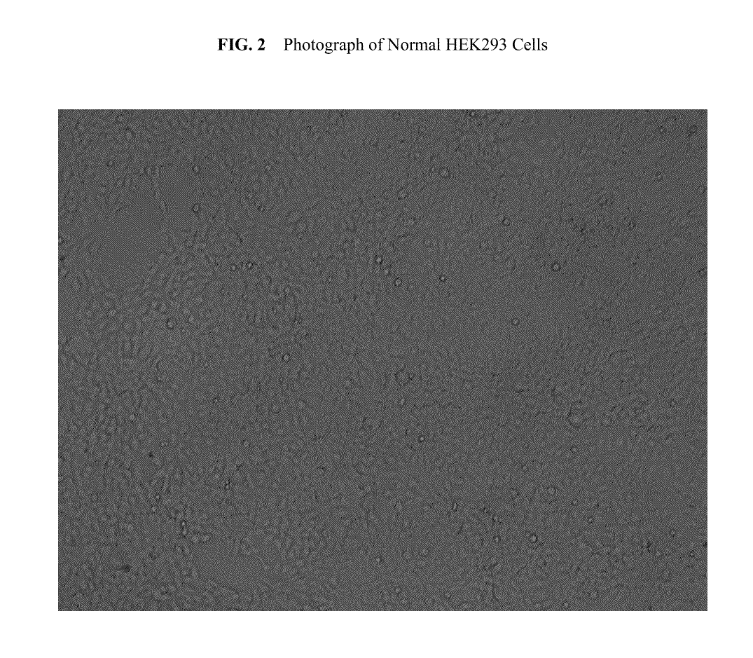

A: Preparation of host cells
Warm water (39° C.) and cell culture flask (75 cm²) were prepared.
Frozen cells were taken out from the nitrogen tank and placed in the warm water. After thawing, cells were centrifuged and the supernatant was removed. Cell pellet was immediately inoculated into cell culture flask containing 20 ml DMEM supplemented with 10% FBS. Cells were cultured in a 37° C. incubator with 5% CO₂.
When monolayer was formed in the 75 cm²-flask (FIG. 2), cells were digested with Trypsin and inoculated into a 175 cm²-flask. When the density reached 90-95%, cells were digested and diluted with 1:3 or 1:4 fresh medium for amplification until a certain amount of cells was achieved.

B: Cell culture
When a certain amount of cells in the 175 cm²-flask was achieved, cells were inoculated into NBS bioreactor for culturing

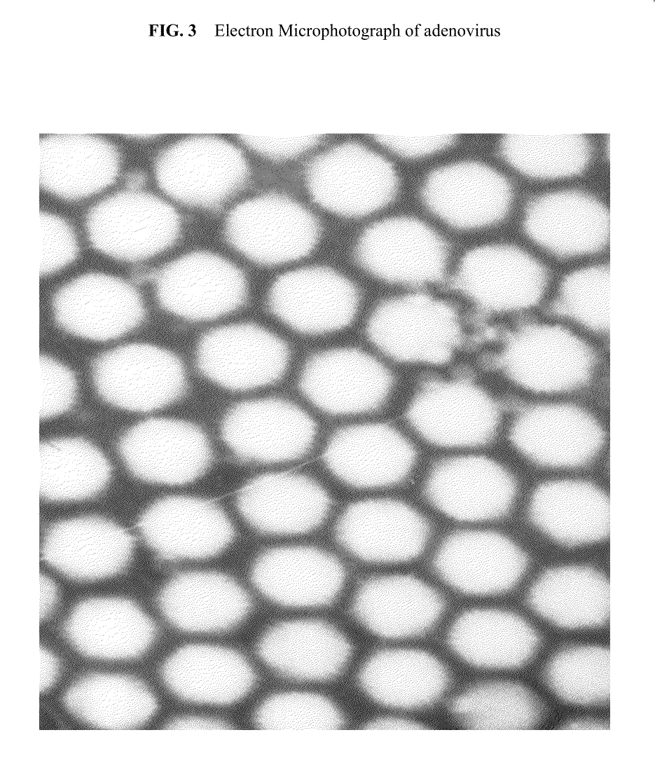

C: Virus culture
When a certain amount of HEK293 cells was achieved, virus was inoculated and cultured (FIG. 3).
According to the titer of virus and host cell density, appropriate quantity of virus was mixed with DMEM containing serum to make virus suspension.
The liquid in the bioreactor was removed and fresh medium was added. Appropriate amount of virus suspension was also added for perfusion culture. Culturing time was controlled at 48-72 hours.

D: Harvesting of virus
HEK293 cells infected with virus were observed and when 95% of the cells were diseased, cells were harvested (FIG. 3).
Pellets were collected after 10 min centrifugation (1000 rpm) at 4° C.
Cell lysis buffer was added to the pellet. The mixture was stored at −80° C.

E: Cell lysis
The cells were lyzed by three times of freezing and thawing (−80° C. or nitrogen←→37° C.).
Supernatant was collected after 30 min centrifugation (4000 rpm) at 4° C.

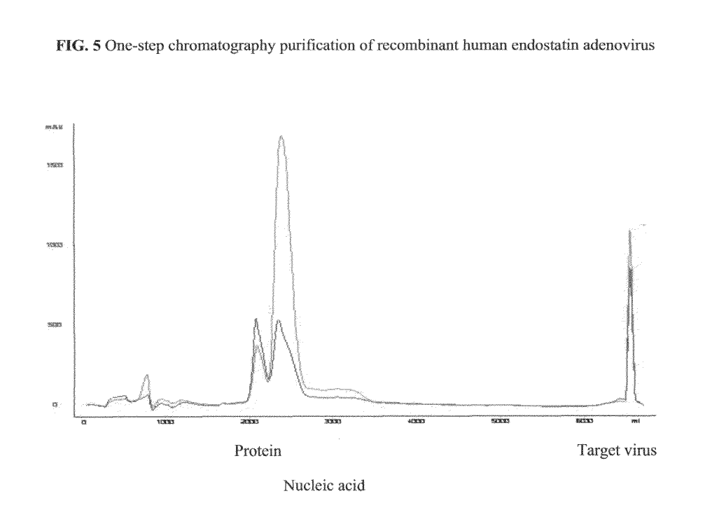

F: Purification of virus
Pretreatment of samples:
   1. Cell lysis: Infected cells were taken out from −80° C. freezer and placed in 37° C. water bath until completely thawed. Three times of freezing and thawing were performed.
   2. Low speed of centrifugation at cold temperature: Lyzed cells were centrifuged (3000 rpm) for 5 min at 4° C.
   3. Treatment with nuclease: After centrifugation, the supernatant was transported to a flask and nuclease was added to remove nucleic acid and reduce the viscosity of the samples. After complete mixing, the samples were placed under 4° C. condition for 90 min.
   4. Filtration: Samples were filtered with a stainless steel filter (two pieces of water-soaked 0.45 μm-membrane was inserted inside the filter). The amount of samples filtered (100-200 mL/time) was dependent on the viscosity of the samples. Filter membrane was changed between each time until filtration was completed.
Chromatography: Virus was purified by one-step anion (Q-Sepharose XL) exchange chromatography
   1. The equipment was turned on for self-examination.
   2. Flowing phase and samples were prepared. All the working solutions and samples must be filtered through a ≦0.45 μm membrane. Buffers were degassed for 10 min using a vacuum.
   3. All the pipelines and chromatography column were cleaned. The chromatography system and columns were cleaned with 0.5 mM NaOH (3 volume of the column) to remove endotoxin. Pure water (5 volume of the column) was used to wash the column until the baseline was stabilized.
   4. Balancing: Buffer A (3 volume of the column) was used to rinse and balance the column. After the column was balanced (once the conductance COND, pH curve was stabilized), “0” was adjusted.
   5. Sample loading: System pump was used to load the samples and the flowing speed was 10-20 mL/min.
   6. Elution: The peaks in the elution were washed to baseline using balancing buffer. Buffer B (0-40%; 3-5 volume of the column) containing 0.1-0.4 mol/L salt was used (flowing speed: 15-25 mL/min) until the baseline was stabilized. The purpose of this procedure was to remove protein and nucleic acid peaks. In addition, the indicators in the samples were completely washed away. Subsequently, the concentration of buffer B was adjusted to 40-100% (containing 0.1-0.4 mol/L salt) to elute. The peaks in the elution were target peaks. Virus was collected when the peaks occurred. Chromatography graphs were shown in FIG. 5.
   7. Virus collection: Virus peaks were collected and concentrated by ultra-filtration. Buffer in the virus solutions was replaced.
   8. Virus samples after ultra-filtration were collected as stock solution.

G: Preparation of injection solutions
Virus crude extractions were filtered through a 0.22 μM membrane to remove bacteria and preservative solution was added. Virus particles were diluted to 1×10¹² VP/mL.

H: Aliquoting of final products
Each vial contains 1 ml virus solution with 1.0×10¹² VP particles.

I: Quality detection of the final products (Table 1)

TABLE 1
Summary of quality detection for the final virus products

| | | | | |
|---|---|---|---|---|
| Physical | Appearance | Pale white | Pale white | Qualified |
| examina- | | suspension | suspension | |
| tion | Quantity in | >=1 ml | 1.02 ml/bottle | Qualified |
| | each bottle | | | |
| Chemical | pH value | 7.8-8.8 | 8.2 | Qualified |
| examina- | | | | |
| tion | | | | |
| Confirma- | Confirmation | Presence of | Positive | Qualified |
| tion | of adenovirus | 880 ± 88 bp | | |
| experi- | vector | band | | |
| ments | Restriction | Consistent | Consistent | Qualified |
| | enzyme | with the | with the | |
| | analysis | standards | standards | |
| | Detection of | Presence of | Positive | Qualified |
| | inserted gene | 0.5 kb ± 50 bp | | |
| | | band | | |
| Purity | Purity | 1.20~1.30 | 1.27 | Qualified |
| detection | detection | | | |
| | (A260/A280 ) | | | |
| | Purity | ≧95% | 98% | Qualified |
| | detection | | | |
| | (HPLC) | | | |
| Titer | Detection of | (1.0 ± 0.1) × | 1.02 × 10¹² | Qualified |
| detection | virus particle | 10¹² VP/vial | VP/vial | |
| | Detection of | ≧3.3 × 10¹⁰ IU/ | 4.6 × 10¹⁰ IU/ | Qualified |
| | virus infection | vial | vial | |
| | titer | | | |
| | Detection of | ≧3.3% | 4.5% | Qualified |
| | ratio titer | | | |
| Activity | Expression | ≧25 ng/ml | 135 ng/ml | Qualified |
| examina- | activity of | | | |
| tion | inserted gene | | | |
| | Biological | ≧250 IU/ml | 4981 IU/ml | Qualified |
| | activity of | | | |
| | inserted gene | | | |
| Residual | Detection of | ≦100 ng/ml | 4.1 ng/ml | Qualified |
| detection | residual | | | |
| | proteins from | | | |
| | HEK293 cells | | | |
| | Detection of | ≦l0 ng/ml | 6.13 ng/ml | Qualified |
| | residual | | | |
| | DNA from | | | |
| | HEK293 cells | | | |
| | Detection of | ≦50 ng/ml | 5.82 ng/mL | Qualified |
| | residual | | | |
| | bovine | | | |
| | proteins | | | |
| Contamina- | Bacterial | Negative | Negative | Qualified |
| tion | detection | | | |
| detection | RCA | ≦1RCA/3 × | Negative | Qualified |
| | detection | 10¹⁰ VP | | |
| | AAV | Absence of | Negative | Qualified |
| | detection | 338 bp ± 34 bp | | |
| | | band | | |
| | Endotoxin | ≦10 EU/ml | ≦0.5 EU/ml | Qualified |
| | detection | | | |

The recombinant human endostatin adenovirus injection solution prepared according to the process disclosed herein was shown to pass the quality control test by the Chinese National Institute for the Control of Pharmaceutical and Biological Products. This product also passed the examination by the Chinese National Center of Drug Trial and an approval was obtained for clinical use.

### Example 2: Inhibitory Effect of Orally Ingested Recombinant Human Endostatin Adenovirus Produced by this Procedure on the Human Transplanted Tumors in Nude Mice and Detection of its in Vivo Activity

### Experiment Materials

A. Cell line:

Nasopharyngeal carcinoma cell line CNE-2 was stored in the Cancer Center of Sun Yat-sen University. Cells were cultured in RPMI medium supplemented with 10% FBS.

B. Experiment animals:

BALB/c nude mice (Gender: female or male; Age: 4-6 week; Weight: 18-24 g) were provided by Animal Center of Sun Yat-sen University (Certificate: No. 26-99A033). Animals were caged in Specific Pathogen Free (SPF) condition (Certificate: No. 26-99S029).

### Experimental Approaches:

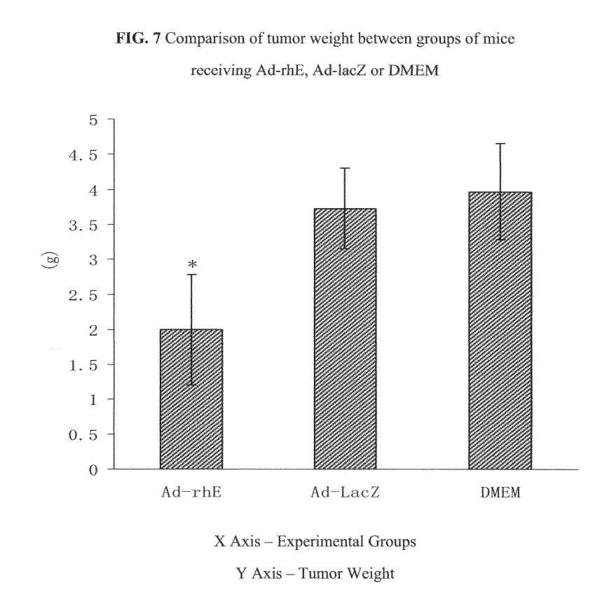

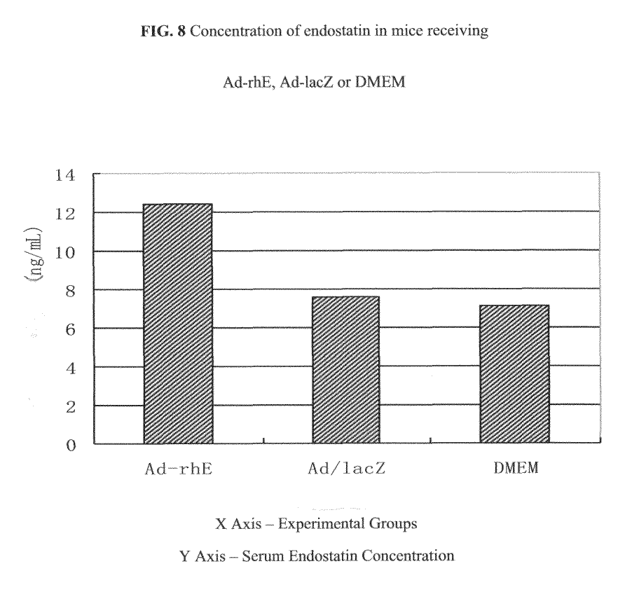

CNE-2 cell suspension (300 μL) containing 1×10⁶ cells was injected subcutaneously into BALB/c female nude mice. Two weeks later, tumors were formed and taken out. After rinsing with sterile normal saline, tumors were cut into small pieces with a diameter of 2-3 cm. Small pieces of tumors were injected subcutaneously into BALB/c female nude mice (age: 4-6 weeks). One week later, the tumor size was increased to a diameter of 6-8 cm and the mice were randomly divided into 3 groups. Experimental group (n=7) was injected with 1×10⁹ pfu recombinant human endostatin adenovirus (Ad-rhE) (100 μL). After 3 days of injection, the tumor size was determined. One week later, Ad-rhE was injected again into the tumors. A total of five times of injection (once per week) was conducted in 5 weeks with a total amount of 5×10⁹ pfu. Adenovirus control group (n=6) was injected with adenovirus-LacZ (Ad-LacZ: vector without human endostatin gene) (1×10⁹ pfu). Blank control group (n=5) was injected with DMEM (100 μL) once a week for a total of 5 weeks. One week after the fifth injection, mice were sacrificed and the tumor size and weight was determined (FIG. 7). The concentration of endostatin in the serum was determined (FIG. 8).

Based on the results shown in FIG. 7 and FIG. 8, expression of endostatin in mice receiving Ad-rhE was higher and sustained longer than that in mice receiving Ad-lacZ or DMEM. In addition, growth of tumor was significantly inhibited in mice receiving Ad-rhE.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance which can be adapted to the practice of this invention in its various embodiments and equivalents thereof.

What is claimed is:

1. A process for producing recombinant human endostatin adenovirus useful for injective administration, the process comprising:
fermenting eukaryotic cells in a culture;
performing adenovirus infection of the eukaryotic cells to yield diseased eukaryotic cells having recombinant human endostatin adenovirus;
harvesting the diseased eukaryotic cells;
causing cell lyses of the diseased eukaryotic cells; and
purifying the resulting recombinant human endostatin adenovirus.

2. The process of claim 1, wherein the eukaryotic cells are Human Embryonic Kidney 293 (HEK293) cells.

3. The process of claim 2, wherein fermenting eukaryotic cells is carried out in a DMEM medium comprising glucose at a concentration greater than about 1 g/L, fetal bovine serum concentration from about 8% to about 12%, and an adenovirus culture medium with about 4% to about 6% serum.

4. The process of claim 1, wherein fermenting is carried out in a NBS bioreactor for cell culture.

5. The process of claim 2, wherein fermenting eukaryotic cells is performed under conditions comprising: at a cell density from about 2×10⁵ mL to about 5×10⁵/mL, at a temperature from about 36° C. to about 37° C., with a CO₂ concentration of about 5%, with a pH in the range from about 7.2 to about 7.4, with an oxygen concentration of about 30% to about 70%, and at a stirring speed of about 30 rpm to about 100 rpm.

6. The process of claim 1, wherein fermenting eukaryotic cells comprises a gradual increase in a medium perfusion rate according to glucose consumption during the culturing process to maintain a concentration of glucose equal to or greater than about 1 g/L.

7. The process of claim 2, wherein adenovirus infection of the eukaryotic cells is carried to a MOI in the range from about 10 to about 30; and harvesting the diseased eukaryotic cells is carried out in about 48 to about 72 hours from virus infection.

8. The process of claim 2, wherein adenovirus infection of the eukaryotic cells is carried out with a pH in the range from about 7.0 to about 7.4, at a temperature from about 36° C. to about 37° C., and with an oxygen concentration of about 30% to about 70%.

9. The process of claim 1, wherein harvesting the diseased eukaryotic cells is carried out when about 95% of the eukaryotic cells have been infected by adenovirus.

10. The process of claim 1, wherein causing cell lyses is by freezing-thawing of the diseased eukaryotic cells.

11. The process of claim 1, wherein purifying the resulting recombinant human endostatin adenovirus comprises:
isolating the human endostatin adenovirus by chromatography; and
separating the human endostatin adenovirus by centrifugation or ultra-filtration.

12. The process of claim 1, further comprising allocating the purified recombinant human endostatin adenovirus into aliquots.

13. The process of claim 11, wherein isolating the human endostatin adenovirus by chromatography comprises using anion exchange fillings selected from Q Sepharose XL Virus Licensed, Q Sepharose XL, DEAE-Sephacel and DEAE-Biogel P for chromatography.

14. The process of claim 13, wherein chromatography is carried out at a pH from about 7.5 to about 8.5.

15. The process of claim 14, wherein chromatography is carried out in a buffer comprising from about 1 mmol/L to about 100 mmol/L of phosphate, a Tris solution, from about 0.1 mmol/L to about 50 mmol/L of MgCl₂, from about 1 mmol/L to about 2000 mmol/L of NaCl, and from about 1% to about 15% of glycerol.

16. The process of claim 13, wherein chromatography is carried out at a sample loading speed from about 10 mL/min to 20 mL/min and an elution speed from about 15 mL/min to about 25 mL/min.

17. The process of claim 11, wherein ultrafiltration is carried out with a 0.22 μM filter.

18. The process of claim 12, wherein the aliquots comprise human endostatin adenovirus at about (1±0.1)×10¹² VP/mL.

19. A process for large-scale production of recombinant human endostatin adenovirus, the process comprising:
fermenting HEK293cells in a culture under conditions of a cell density from about 2×10⁵ mL to about 5×10⁵/mL, a temperature from about 36° C. to about 37° C., a CO₂ concentration of about 5%, a pH in the range from about 7.2 to about 7.4, an oxygen concentration of about 30% to about 70%, and a stirring speed of about 30 rpm to about 100 rpm;
performing adenovirus infection of HEK293 cells to yield diseased eukaryotic cells having recombinant human endostatin adenovirus, wherein the adenovirus infection is carried out to a MOI in the range from about 10 to about 30 and under conditions of a pH in the range from about 7.0 to about 7.4, a temperature from about 36° C. to about 37° C., and an oxygen concentration of about 30% to about 70%;
harvesting the diseased HEK293 cells in about 48 to about 72 hours from virus infection;
causing cell lyses by at least three cycles of freezing-thawing of the diseased HEK293 cells;
isolating the human endostatin adenovirus by anion exchange chromatography carried out at a pH from about 7.5 to about 8.5 with a buffer comprising from about 1 mmol/L to about 100 mmol/L of phosphate, a Tris solution, from about 0.1 mmol/L to about 50 mmol/L of MgCl₂, from about 1 mmol/L to about 2000 mmol/L of NaCl, and from about 1% to about 15% of glycerol;
separating the human endostatin adenovirus by ultra-filtration with a 0.22 μM filter; and
allocating the purified recombinant human endostatin adenovirus into aliquots comprising human endostatin adenovirus from about 1.0×10¹² to about 3.0×10¹² VP/mL.

20. The process of claim 19, wherein anion exchange chromatography uses fillings selected from Q Sepharose XL Virus Licensed, Q Sepharose XL, DEAE-Sephacel and DEAE-Biogel P for chromatography.
